(19) United States
(12) Plant Patent Application Publication
(10) Pub. No.: US 2026/0293786 P1
(43) Pub. Date: Sep. 24, 2026

(54) Colocasia plant named 'Tunpuran'

(50) ;

(71) Applicant: Preecha Tunok, Phetchabun (TH)

(72) Inventor: Preecha Tunok, Phetchabun (TH)

(21) Appl. No.: 19/551,286

(22) Filed: Feb. 26, 2026

(30) Foreign Application Priority Data
Mar. 18, 2025 (QZ) 2025/0492

(51) Int. Cl. A01H 6/10; A01H 5/12

(52) U.S. Cl. CPC A01H 6/10; A01H 5/12
USPC PLT/373

(57) ABSTRACT

A new and distinct Colocasia plant named ‘Tunpuran’ particularly distinguished by: dark colored foliage, with mature leaves displaying a near-black coloration on the upper surface; slightly leathery and velvety texture with a matte luster on the upper leaf surface; strong greyed-purple venation coloration; a midrib and radiating primary and secondary veins strongly colored and sharply contrasting with the dark leaf blade, forming a pronounced and stable vein pattern that remains visible even on fully mature leaves; leaves displaying a strong greyed-purple coloration around the basal sinus and midrib region; large, peltate, broad ovate to cordate shaped leaves with a broad blade and well-defined basal lobes; foliage presented in an upright to slightly outward-facing manner; petioles colored a dark greyed-purple and brown; and an upright, clumping growth habit suitable for ornamental use in containers and landscape plantings.

Genus and species: Colocasia esculenta (L.) Schott.

Variety denomination: ‘Tunpuran’.

### CROSS-REFERENCE TO A RELATED APPLICATION

This application claims priority to European Community Plant Variety Office (CPVO) Plant Breeder's Rights application no. 2025/0492 filed on Mar. 18, 2025, the entire contents of which is incorporated by reference herein. The information in the CPVO application, including the disclosed first sale of the claimed plant on Apr. 15, 2024 in the Netherlands, originated directly from the Inventor. The Inventor claims a prior art exemption under 35 U.S.C. 102(b)(1) for disclosures and/or sales that fall within a one-year grace period prior to the effective filing date of this application.

### BACKGROUND OF THE NEW PLANT

The present invention comprises a new and distinct variety of Colocasia plant, botanically known as Colocasia esculenta, and hereinafter referred to by the variety name ‘Tunpuran’. The new Colocasia cultivar ‘Tunpuran’ is a seedling selection that resulted from a controlled cross pollination breeding program carried out by the inventor in April 2022 in a greenhouse located in Phetchabun, Thailand. The new variety originated as a seedling from crossing a proprietary unnamed and unpatented Colocasia breeding line as the seed parent, with a proprietary unnamed and unpatented Colocasia breeding line as the pollen parent. From said cross, 400 seeds were harvested, then germinated, and the resulting seedlings were grown to a mature size in order to evaluate for desirable commercial characteristics. From amongst these seedlings, ‘Tunpuran’ was selected by the inventor in June 2023 for further asexual propagation and commercialization based on its unique foliage colorations, fast growth rate, and the ability to produce suckers (a common term used to refer to vegetative offsets that can alone, when removed from the main plant, are capable of reproducing a new plant).

The new Colocasia cultivar ‘Tunpuran’ was first reproduced asexually in June 2023 by suckers in Phetchabun, Thailand. Subsequently, ‘Tunpuran’ was introduced into meristematic tissue culture for further asexual propagation. Asexual propagation of the new variety has shown that the unique features of the new variety are stable and reproduced true-to-type to date through 8 successive generations.

### SUMMARY OF THE INVENTION

The new Colocasia cultivar ‘Tunpuran’ has not been observed under all possible environmental conditions. The phenotype may vary somewhat with variations in environment such as temperature, day length, light intensity, water status, fertilizer rate and type, without, however, any variance in genotype.

The following are the most outstanding and distinguishing characteristics of this new Colocasia cultivar. The combination of these characteristics distinguishes ‘Tunpuran’ as a new and distinct variety of Colocasia.
1. Colocasia ‘Tunpuran’ exhibits very dark colored foliage, with mature leaves displaying a near-black coloration on the upper surface.
2. Colocasia ‘Tunpuran’ exhibits a distinctive slightly leathery and velvety texture with a matte luster on the upper leaf surface, giving the foliage a soft, fabric-like appearance.
3. Colocasia ‘Tunpuran’ exhibits a strong greyed-purple venation coloration on its mature leaves. The central midrib and radiating primary and secondary veins are strongly colored and sharply contrast with the dark leaf blade, forming a pronounced and stable vein pattern that remains visible on fully mature leaves.
4. Colocasia ‘Tunpuran’ exhibits leaves displaying a strong greyed-purple coloration around the basal sinus and midrib region.
5. Colocasia ‘Tunpuran’ exhibits large, peltate, broad ovate to cordate shaped leaves with a broad blade and well-defined basal lobes. The foliage is presented in an upright to slightly outward-facing manner.
6. Colocasia ‘Tunpuran’ exhibits petioles colored a dark greyed-purple and brown.
7. Colocasia ‘Tunpuran’ exhibits an upright, clumping growth habit suitable for ornamental use in containers and landscape plantings.

### DESCRIPTION OF THE PHOTOGRAPHS

This new Colocasia cultivar is illustrated by the accompanying-colored photographs which show the overall appearance and distinct characteristics of the plant. The colors shown are as true as can be reasonably obtained by conventional photographic procedures. The photographs are 6-month-old plants grown in 19-centimeter circular containers grown in a greenhouse in Nootdorp, The Netherlands. Colors in the photographs may differ slightly from the color values cited in the botanical description which accurately describes the colors of the new variety.

FIG. 1. shows the overall plant form and foliage of ‘Tunpuran’.

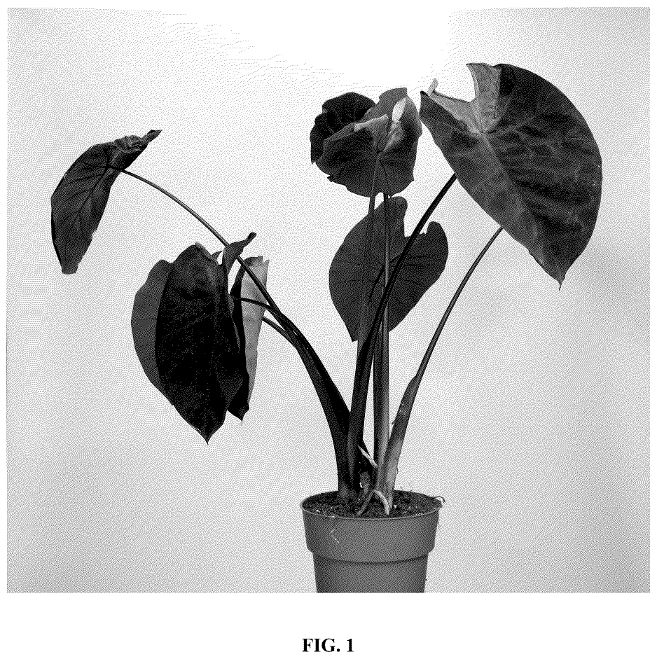

FIG. 2. shows a close-up of a typical fully opened juvenile leaf of ‘Tunpuran’.

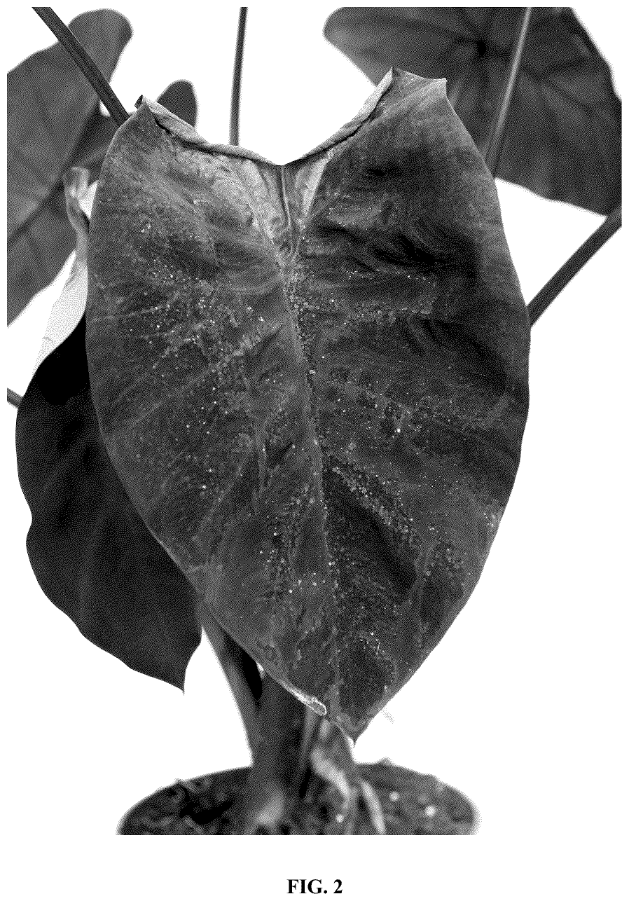

FIG. 3. shows a close-up of a typical mature leaf of ‘Tunpuran’.

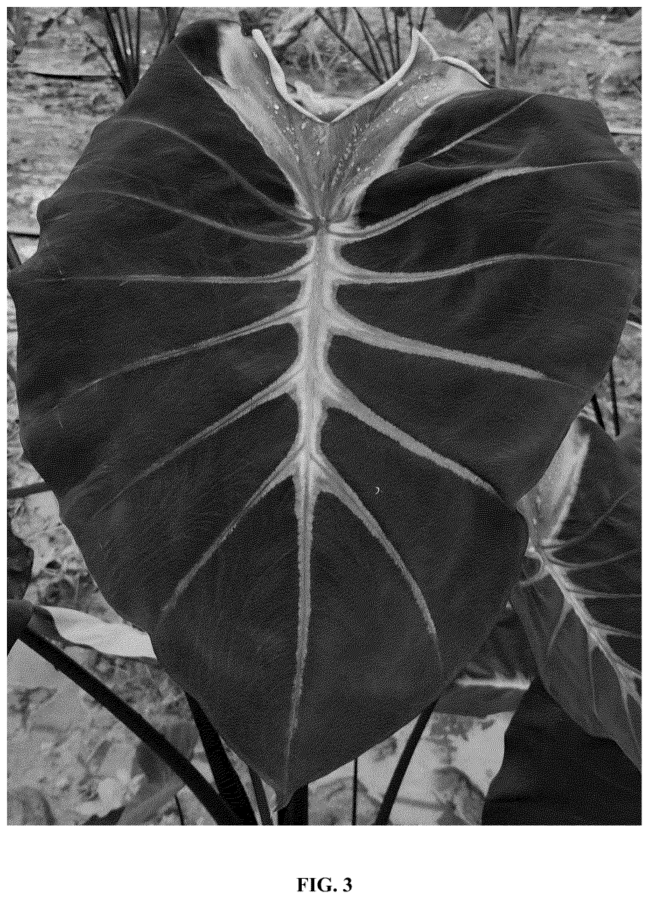

### DESCRIPTION OF THE NEW VARIETY

In the following description, color references are made to The Royal Horticultural Society Colour Chart, Sixth Edition, except where general color terms of ordinary dictionary significance are used.

The following observations and measurements describe plants grown in Nootdorp, The Netherlands in a conventional greenhouse with basic heating and without shade. Growing conditions consisted of average day temperatures of 10° C. to 25° C. and average night temperatures of 10° C. to 18° C. Plants were grown under natural light levels with no artificial lighting. Detailed descriptions were taken in September 2025 from 6-month-old plants grown in 19-centimeter circular containers. Measurements and numerical values represent averages of typical plant types.

### DETAILED BOTANICAL DESCRIPTION

Classification:
   Family.—Araceae.
   Botanical: Colocasia esculenta.
   Common name.—Taro or Elephant Ears.
   Denomination.—‘Tunpuran’.
General description:
   Plant type.—Tropical herbaceous perennial.
   Crop time.—Approximately 26 weeks from a tissue-cultured plantlet to a saleable plant in a 19.0-centimeter circular container.
   Overall plant shape.—Inverted triangular.
   Growth habit.—Stemless, upright with leaves emerging in basal clumps.
   Height from soil level to top of foliar plane.—Average 81.3 cm.
   Plant diameter or area of spread.—Average 64.3 cm.
   Growth rate.—Moderate.
   Plant vigor.—High.
   Branching habit.—Leaves in basal clumps, no stems present.
   Number of basal clumps per plant.—3.
   Roots.—Moderately fleshy, non-fibrous and very fine, long roots having an average length of 45.0 cm and an average width of 0.2 mm to 0.9 mm, colored near 184C and 184D (greyed-purple).
   Corms.—No corms have been observed to date.
Foliage description:
   Arrangement.—Alternate.
   Quantity of leaves per basal clump.—Average of 4.
   Lamina.—Shape: Peltate, broad ovate to cordate. Aspect: Slightly convex to convex. Length: Average 25.8 cm. Width: Average 17.6 cm. Apex shape: Abruptly acute. Base shape: Cordate to sagittate with rounded lobes. Margins: Entire. Margin undulation: Shallow and coarsely undulate. Texture: Upper surface: Slightly leathery and velvety. Lower surface: Slightly leathery. Pubescence: Not present. Rugosity (both upper and lower surfaces): Non-rugose. Luster (both upper and lower surfaces): Matte. Color: Juvenile foliage: Upper surface: Upon opening: 132A (dark green); inner half of basal lobes from the basal notch to the base of the lobes colored near 145A (yellow-green) and tinged near 197C (greyed-green); areas adjacent to the secondary veins colored near NN137C (green). When fully opened: Juvenile leaves transition to near 202A (black) with the inner half of the basal lobes transitioning to 187A (greyed-purple). Lower surface: Near N200B (brown), tinged N187C (greyed-purple), and fading to near N200B towards the primary vein and secondary veins; inner half of basal lobes from the basal notch to the base of the lobes colored near 145A (yellow-green) and tinged near 199C (grey-brown). Mature foliage: Upper surface: Near 203B (black); inner half of basal lobes from the basal notch to the base of the lobes near 186A (purplish red) and near 186C to 186D (purplish pink); areas flanking the primary vein near 186C to 186D (purplish pink); areas flanking the secondary veins near a blend of 186A (purplish red) and N187A (purplish grey). Lower surface: A blend of near N187A and N187B (greyed-purple) and fading to near N200B (brown) towards the primary vein and secondary veins; inner half of basal lobes from the basal notch to the base of the lobes colored near N186C (greyed-purple). Venation: Pattern: Pinnate and slightly furrowed. Color (juvenile foliage): Upper surface: Primary vein colored near a blend of 186C (greyed-purple) and 201C (grey); secondary veins colored near N189A (greyed-green). Lower surface: Primary vein colored near 183B (greyed-purple); secondary veins colored near 177C (greyed-orange). Color (mature foliage): Upper surface: Primary vein near 186B (purplish red) to 186C (purplish pink); secondary veins near a blend of N186A (purplish grey) and 203C (black). Lower surface: Primary vein and secondary veins colored near N186C (greyed-purple).
   Petiole.—Length: Average 52.5 cm. Diameter: Proximal end: Average 1.8 cm. Distal end: Flattened; average 0.4 cm at the widest, horizontal point and an average of 0.3 cm at the narrowest, vertical point. Texture: Smooth and glabrous. Luster: Moderately glossy. Strength: Medium strong. Color: Upper surface: A blend of near N186C (greyed-purple) and 200A (brown) at the proximal end and near N186C at the distal end. Lower surface: Near 200B (brown) and 200C (brown) at the proximal end and near 187C (greyed-purple) at the distal end. Wings: Length: Average 24.7 cm. Width: Average 1.2 cm. Color: Upper surface: A blend of near 176B (greyed-orange) and 183C (greyed-purple). Lower surface: 200B (brown).
Inflorescence: None observed to date.
High temperature tolerance: Up to 40 degrees Celsius.
Low temperature tolerance: At least hardy to USDA zone 10.
USDA hardiness zones: Zones 7 to 10.
Rain tolerance: Moderate to high.
Wind tolerance: Low.
Disease and pest tolerance: No claims are made to any superior disease or insect resistance or tolerance.

### COMPARISON WITH THE PARENT PLANTS

The new Colocasia cultivar ‘Tunpuran’ can be distinguished from its unnamed and unpatented female or seed parent plant in the following characteristics described in Table 1 below.

TABLE 1

| Characteristic | ‘Tunpuran’ | Female Parent |
|---|---|---|
| Mature leaf | Very dark purple | Medium to dark green |
| coloration. | to near-black | lamina with contrasting |
| | lamina. | white or cream markings. |
| Leaf surface | Velvety and matte | Smooth, semi-glossy to |
| texture and | with a soft, | glossy. |
| luster. | non-glossy | |
| | appearance. | |
| Primary | Bright greyed-purple | Cream-white to pale |
| venation color. | veins strongly | veins integrated with |
| | contrasting with | a white central band. |
| | the dark lamina. | |
| Overall color | Dark lamina | Green lamina with a |
| contrast. | with vivid greyed- | broad creamy-white |
| | purple venation in | central band extending |
| | a radiating pattern. | into veins. |
| Appearance of | Predominantly dark, | Variegated foliage |
| foliage. | dramatic foliage | characterized by |
| | with bright greyed- | white center markings |
| | purple venation | rather than dark |
| | as the dominant | coloration. |
| | ornamental trait. | |

The new Colocasia cultivar ‘Tunpuran’ can be distinguished from its unnamed and unpatented male or pollen parent plant in the following characteristics described in Table 2 below.

TABLE 2

| Characteristic | ‘Tunpuran’ | Male Parent |
|---|---|---|
| Mature leaf | Very dark purple to | Dark purple to black |
| coloration. | near-black lamina. | lamina, generally |
| | | uniform in color. |
| | | without contrasting |
| | | venation |
| Leaf surface | Velvety and matte | Smooth, glossy leaf |
| texture. | with a soft, non- | surface lacking a |
| | glossy appearance. | velvety texture. |
| Primary | Bright greyed-purple | Dark purple to black, |
| venation color. | veins strongly | typically blending with |
| | contrasting with the | the lamina and lacking |
| | dark lamina. | bright contrast. |
| Ornamental | Strong bicolored | Mostly solid dark |
| pattern. | effect created by vivid | foliage with minimal |
| | greyed-purple venation | contrasting pattern. |
| | over a dark background | |
| | lamina coloration. | |

### COMPARISON WITH RELATED KNOWN VARIETY

The new Colocasia cultivar ‘Tunpuran’ can be distinguished from the related known variety of Colocasia esculenta cultivar ‘Corede’ (U.S. Plant Pat. No. 34,729) in the following characteristics described in Table 3 below.

TABLE 3

| Characteristic | ‘Tunpuran’ | ‘Corede’ |
|---|---|---|
| Leaf surface | Velvety and matte | Smooth to slightly |
| texture. | with a soft, non- | glossy leaf surface |
| | glossy appearance. | lacking a velvety |
| | | texture. |
| Mature leaf | Very dark purple to | Dark burgundy to |
| coloration. | near black. | purple generally |
| | | lighter than near- |
| | | black. |
| Intensity of | Bright, greyed-purple | Red to pink venation, |
| venation color. | primary and secondary | typically less vivid and |
| | veins with strong | with lower contrast. |
| | contrast. | |
| Mature leaf | High contrast between | Venation contrast tends |
| color stability. | dark lamina and bright | to reduce as leaves |
| | greyed-purple venation | mature. |
| | maintained on mature | |
| | leaves. | |
| Petiole. | Dark greyed-purple and | Dark red to burgundy |
| | brown colored petioles. | petioles, generally |
| | | lighter than those of |
| | | ‘Tunpuran’ |

What is claimed is:

1. A new and distinct cultivar of Colocasia plant named ‘Tunpuran’, substantially as illustrated and described herein.
